(19) United States
(12) Patent Application Publication
(10) Pub. No.: US 2004/0265980 A1
(43) Pub. Date: Dec. 30, 2004

(54) Use of POX4 promoter to increase gene expression in Candida tropicalis

(76) Inventors: Yeyan Zhang, Mason, OH (US); C. Ron Wilson, Loveland, OH (US); David L. Craft, Ft. Thomas, KY (US); L. Dudley Eirich, Milford, OH (US); Robert W. Frayer, Middletown, OH (US)

Correspondence Address: COGNIS CORPORATION, PATENT DEPARTMENT, 300 BROOKSIDE AVENUE, AMBLER, PA, 19002, US

(21) Appl. No.: 10/629,266

(22) Filed: Jul. 29, 2003

Related U.S. Application Data
(60) Provisional application No. 60/401,212, filed on Aug. 5, 2002.

(51) Int. Cl. C12P 7/64; C12P 7/40; C12N 1/18; C12N 15/74

(52) U.S. Cl. 435/134; 435/136; 435/254.2; 435/483

(57) ABSTRACT

The present invention provides methods of converting or increasing conversion of a fatty acid to its corresponding dicarboxylic acid. The methods comprise isolating a promoter from a yeast gene which gene is induced when the yeast is grown on a fatty acid or alkane substrate, and operably linking the promoter to a gene involved in dicarboxylic acid production to form an expression vector. Yeast cells are subsequently transformed with such an expression vector and cultured in a media containing an organic substrate biooxidizable to a mono- or polycarboxylic acid, and resultant yeast cells convert or increase conversion of fatty acids to their corresponding dicarboxylic acids. Examples of promoters that may be used in the methods of the present invention include those from C. tropicalis catalase, citrate synthase, 3-ketoacyl-CoA thiolase A, citrate synthase, O-acetylhomserine sulphydrylase, protease, carnitine O-acetyltransferase, hydratase-dehydrogenase, and epimerase genes. A preferred promoter for use in a subject expression vector is the POX4 gene promoter. Examples of genes involved in dicarboxylic acid production include members of an ω-hydroxylase complex such as e.g., CYP, NCP, or CYTb5 genes. Host cells comprising such expression vectors are also provided. Preferred host cells are include Yarrowia, Candida, Bebaromyces, Saccharomyces, Schizosaccharomyces, and Pichia.

### CROSS-REFERENCE TO RELATED APPLICATION

This application claims benefit from U.S. Provisional Application Ser. No. 60/401,212, filed Aug. 5, 2002, which application is incorporated herein by reference.

### BACKGROUND OF THE INVENTION

Aliphatic dioic acids are versatile chemical intermediates useful as raw materials for the preparation of perfumes, polymers, adhesives and macrolid antibiotics. While several chemical routes to the synthesis of long-chain α, ω-dicarboxylic acids are available, the synthesis is not easy and most methods result in mixtures containing shorter chain lengths. As a result, extensive purification steps are necessary. While it is known that long-chain dioic acids can also be produced by microbial transformation of alkanes, fatty acids or esters thereof, chemical synthesis has remained the most commercially viable route, due to limitations with the current biological approaches.

Several strains of yeast are known to excrete α, ω-dicarboxylic acids as a byproduct when cultured on alkanes or fatty acids as the carbon source. In particular, yeast belonging to the Genus Candida, such as C. albicans, C. cloacae, C. guillermondii, C. intermedia, C. ipolytica, C. maltosa, C. parapsilosis and C. zeylenoides are known to produce such dicarboxylic acids (Agr. Biol. Chem. 35: 2033-2042 (1971)). Also, various strains of C. tropicalis are known to produce dicarboxylic acids ranging in chain lengths from C₁₁ through C₁₈ (Okino et al., B M Lawrence, B D Mookherjee and B J Willis (eds), in Flavors and Fragrances: A World Perspective. Proceedings of the 10th International Conference of Essential Oils, Flavors and Fragrances, Elsevier Science Publishers BV Amsterdam (1988)), and are the basis of several patents as reviewed by Bühler and Schindler, in Aliphatic Hydrocarbons in Biotechnology, H. J. Rehm and G. Reed (eds), Vol. 169, Verlag Chemie, Weinheim (1984).

Studies of the biochemical processes by which yeasts metabolize alkanes and fatty acids have revealed three types of oxidation reactions: α-oxidation of alkanes to alcohols, ω-oxidation of fatty acids to α, ω-dicarboxylic acids and the degradative β-oxidation of fatty acids to CO₂ and water. The first two types of oxidations are catalyzed by microsomal enzymes while the last type takes place in the peroxisomes. In C. tropicalis, the first step in the ω-oxidation pathway is catalyzed by a membrane-bound enzyme complex (ω-hydroxylase complex) including a cytochrome P450 monooxygenase and a NADPH dependent cytochrome reductase. This hydroxylase complex is responsible for the primary oxidation of the terminal methyl group in alkanes and fatty acids as described, e.g., in Gilewicz et al., Can. J. Microbiol. 25:201 (1979), incorporated herein by reference. The genes which encode the cytochrome P450 and NADPH reductase components of the complex have previously been identified as P450ALK and P45ORED respectively, and have also been cloned and sequenced as described, e.g., in Sanglard et al., Gene 76:121-136 (1989), incorporated herein by reference. P450ALK has also been designated P450ALK1. More recently, ALK genes have been designated by the symbol CYP and RED genes have been designated by the symbol CPR. See, e.g., Nelson, Pharmacogenetics 6(1):1-42 (1996), which is incorporated herein by reference. See also Ohkuma et al., DNA and Cell Biology 14:163-173 (1995), Seghezzi et al., DNA and Cell Biology, 11:767-780 (1992) and Kargel et al., Yeast 12:333-348 (1996), each incorporated herein by reference. In addition, CPR genes are now also referred to as NCP genes. See, e.g., De Backer et al., Antimicrobial Agents and Chemotherapy, 45:1660 (2001). For example, P450ALK is also designated CYP52 according to the nomenclature of Nelson, supra. Fatty acids are ultimately formed from alkanes after two additional oxidation steps, catalyzed by alcohol oxidase as described, e.g., in Kemp et al., Appl. Microbiol. and Biotechnol. 28: 370-374 (1988), incorporated herein by reference, and aldehyde dehydrogenase. The fatty acids can be further oxidized through the same or similar pathway to the corresponding dicarboxylic acid. The ω-oxidation of fatty acids proceeds via the ω-hydroxy fatty acid and its aldehyde derivative, to the corresponding dicarboxylic acid without the requirement for CoA activation. However, both fatty acids and dicarboxylic acids can be degraded, after activation to the corresponding acyl-CoA ester through the β-oxidation pathway in the peroxisomes, leading to chain shortening. In mammalian systems, both fatty acid and dicarboxylic acid products of ω-oxidation are activated to their CoA-esters at equal rates and are substrates for both mitochondrial and peroxisomal β-oxidation (J. Biochem., 102:225-234 (1987)). In yeast, β-oxidation takes place solely in the peroxisomes (Agr. Biol. Chem. 49:1821-1828 (1985)).

Cytochrome P450 monooxygenases (P450s) are terminal monooxidases of a multicomponent enzyme system including P450 and CPR (NCP). In some instances, a second electron carrier, cytochrome b5(CYTb5) and its associated reductase are involved as described below and in Morgan, et al., Drug Metab. Disp. 12:358-364 (1984). The P450s comprise a superfamily of proteins which exist widely in nature having been isolated from a variety of organisms as described e.g., in Nelson, supra. These organisms include various mammals, fish, invertebrates, plants, mollusk, crustaceans, lower eukaryotes and bacteria (Nelson, supra). First discovered in rodent liver microsomes as a carbon-monoxide binding pigment as described, e.g., in Garfinkel, Arch. Biochem. Biophys. 77:493-509 (1958), which is incorporated herein by reference, P450s were later named based on their absorption at 450 nm in a reduced-CO coupled difference spectrum as described, e.g., in Omura et al., J. Biol. Chem. 239:2370-2378 (1964), which is incorporated herein by reference.

Monooxygenation reactions catalyzed by cytochromes P450 in a eukaryotic membrane-bound system require the transfer of electrons from NADPH to P450 via NADPH-cytochrome P450 reductase (CPR) as described, e.g., in Taniguchi et al., Arch. Biochem. Biophys. 232:585 (1984), incorporated herein by reference. CPR is a flavoprotein of approximately 78,000 Da containing 1 mol of flavin adenine dinucleotide (FAD) and 1 mol of flavin mononucleotide (FMN) per mole of enzyme as described, e.g., in Potter et al., J. Biol. Chem. 258:6906 (1983), incorporated herein by reference. The FAD moiety of CPR is the site of electron entry into the enzyme, whereas FMN is the electron-donating site to P450 as described, e.g., in Vermilion et al., J. Biol. Chem. 253:8812 (1978), incorporated herein by reference. The overall reaction is as follows:

H⁺+RH+NADPH+O₂→ROH+NADP⁺+H₂O

Binding of a substrate to the catalytic site of P450 apparently results in a conformational change initiating electron transfer from CPR to P450. Subsequent to the transfer of the first electron, O₂ binds to the Fe₂⁺-P450 substrate complex to form Fe₃⁺-P450-substrate complex. This complex is then reduced by a second electron from CPR, or, in some cases, NADH via a second electron carrier, cytochrome b5 (CYTb5) and its associated NADH-cytochrome b5 reductase as described, e.g., in Guengerich et al., Arch. Biochem. Biophys. 205:365 (1980), incorporated herein by reference, and Morgan, supra. Most of the aforementioned studies implicate CYTb5 as being involved in the pathway only for the transfer of the second electron. One atom of this reactive oxygen is introduced into the substrate, while the other is reduced to water. The oxygenated substrate then dissociates, regenerating the oxidized form of the cytochrome P450 as described, e.g., in Klassen, Amdur and Doull, Casarett and Doull's Toxicology, Macmillan, New York (1986), incorporated herein by reference. With respect to the CYTb5, several other models of the role of this protein in P450 expression have been proposed besides its role as an electron carrier.

While several chemical routes to the synthesis of long-chain α,ω.-dicarboxylic acids as 9-octadecenedioic acid are available, such methods are complex and usually result in mixtures containing shorter chain lengths. As a result, extensive purification steps are necessary. As an alternative to chemical syntheses, long chain α,ω-dicarboxylic acids such as 9-octadecenedioic acid can be made via fermentation methods such as microbial transformation of the corresponding hydrocarbons such as alkanes or alkenes, fatty acids or esters thereof One method for producing substantially pure α,ω-dicarboxylic acids in substantially quantitative yield is described in U.S. Pat. No. 5,254,466, the entire contents of which are incorporated herein by reference. This method comprises culturing a C. tropicalis strain wherein both copies of the chromosomal POX5 and each of the POX4A and POX4B genes are disrupted in a culture medium containing a nitrogen source, an organic substrate and a cosubstrate.

The POX4 and POX5 gene disruptions effectively block the β-oxidation pathway at its first reaction (which is catalyzed by acyl-CoA oxidase) in a C. tropicalis host strain. The POX4A and POX5 genes encode distinct subunits of long chain acyl-CoA oxidase, which are the peroxisomal polypeptides (PXPs) designated PXP-4 and PXP-5, respectively. The disruption of one or more of these genes results in a partial or complete inactivation of the β-oxidation pathway thus allowing enhanced yields of dicarboxylic acid by redirecting the substrate toward the α-oxidation pathway and also prevents reutilization of the dicarboxylic acid products through the β-oxidation pathway.

Another method for producing substantially pure α,ω-dicarboxylic acids in substantial yield is described in U.S. Pat. No. 6,331,420, the entire content thereof incorporated by reference herein as if fully set forth. This method includes increasing the CYP and CPR (NCP) enzymes by amplification of the CYP and CPR gene copy number in a C. tropicalis strain, and culturing the genetically modified strain in media containing an organic substrate.

Gene(s) involved in the bioconversion of various feed stocks, e.g., HOSFFA (high oleic sunflower oil, i.e., fatty acid mixtures containing oleic acid commercially available from Cognis Corp. as Edenor® and Emersol®), have native promoters that control their transcriptional regulation. These promoters are sometimes inadequate to achieve the level of transcription needed to make a gene(s) product, e.g., CYP, CPR or CYTb5, that is involved in a given process.

Accordingly, there exists a need for improved processes for increasing dicarboxylic acid production in yeast.

### SUMMARY OF THE INVENTION

In accordance with the present invention, it has been surprisingly found that the POX4 promoter is strongly induced even in those yeast strains where the POX4 gene is disrupted. Results indicate that a cryptic protein product is produced which is not functional. The present invention is therefore directed to use of the POX4 gene promoter as well as other promoters from other alkane or fatty acid inducible genes, for the expression of heterologous genes in yeast cells.

Thus, the present invention involves improved processes and compositions for increasing dicarboxylic acid production in a microorganism such as yeast. In one embodiment, dicarboxylic acid production is increased by isolating a gene involved in dicarboxylic acid production having a weak promoter and replacing the weak promoter with a strong, inducible promoter from a yeast gene having a high level of expression. The substitution of a strong, inducible promoter operably linked to a target gene involved in dicarboxylic acid production increases the level of transcription of that target gene.

Promoters which are particularly useful for the practice of the present invention include those which are inducible in yeasts grown on alkanes or fatty acids as substrate. Thus, for example, useful promoters include but are not limited to those of the following Candida tropicalis genes: catalase, citrate synthase, 3-ketoacyl-CoA thiolase A, citrate synthase, O-acetylhomoserine sulphydrylase, protease, carnitine O-acetyltransferase, hydratase-dehydrogenase, epimerase, and acyl-CoA oxidase.

In accordance with the present invention, there are provided nucleic acid sequences comprising a POX4 gene promoter from Candida tropicalis operably linked to the open reading frame of a gene encoding a heterologous protein. For example, the heterologous protein may be a protein from a pathway that uses fatty acids or alkanes as substrate. Preferably, the heterologous protein is a member of an ω-hydroxylase complex. Examples of heterologous proteins which are members of the ω-hydroxylase complex include, but are not limited to CYP, NCP and cytochrome b5. Genes corresponding to these proteins include, but are not limited to CYP52A2A, CYP52A5A, NCP1B, and CYTb5.

The present invention also provides an expression vector comprising a nucleic acid sequence including a POX4 gene promoter operably linked to the open reading frame of a gene encoding a heterologous protein. Preferably, the heterologous protein is a member of an ω-hydroxylase complex. Expression vectors in accordance with the present invention include for example, plasmids, phagemids, phage, cosmids, yeast artificial chromosomes or linear DNA vectors. Examples of plasmids include but are not limited to e.g, yeast episomal plasmids or yeast replication plasmids.

In another aspect of the invention, a process for transforming a host cell is provided which includes isolating a POX4 promoter; isolating a target gene; operably linking a POX4 promoter to the open reading frame target gene to create a fusion gene; inserting the fusion gene into an expression vector; and transforming the host cell with the expression vector. Preferably, the target gene codes for a member of an ω-hydroxylase complex.

Also provided by the present invention is a host cell comprising a nucleic acid sequence including a POX4 gene promoter operably linked to the open reading frame of a gene encoding a heterologous protein such as a member of an ω-hydroxylase complex. Examples of such genes include CYP52A2A, CYP52A5A, NCP1B, and CYTb5. Examples of host cells include cells from Yarrowia, Candida, Bebaromyces, Saccharomyces, Schizosaccharomyces, or Pichia. Preferably, the host cell is from Candida such as e.g., a host cell from C. tropicalis, C. maltosa, C. apicola, C. paratropicalis, C. albicans, C. cloacae, C. guiillermondii, C. intermedia, C. lipolytica, C. parapsilosis, or C. zeylenoides. Most preferably, the host cell is from C. tropicalis.

In another aspect of the invention, there is provided a method of converting a fatty acid to its corresponding dicarboxylic acid. The method comprises the steps of isolating a promoter from a yeast gene which is induced when the yeast is grown on fatty acids or alkanes; isolating a target gene involved in dicarboxylic acid production; operably linking the inducible gene promoter to the open reading frame (ORF) of the target gene involved in dicarboxylic acid production to create a fusion gene; inserting the fusion gene into an expression vector; transforming a yeast host cell with the expression vector; and culturing the transformed yeast host cell in a media containing an organic substrate that is biooxidizable to a mono- or polycarboxylic acid. Preferably, the promoter is the POX4 promoter or else a promoter isolated from a C. tropicalis gene including but not limited to, a catalase, citrate synthase, 3-ketoacyl-CoA thiolase A, citrate synthase, O-acetylhomoserine sulphydrylase, protease, carnitine O-acetyltransferase, hydratase-dehydrogenase, or epimerase gene. Preferably, the target gene encodes a member of an ω-hydroxylase complex such as any of the CYP, NCP, or CYTb5 genes.

In still another aspect of the invention, there is provided a method of converting a fatty acid to its corresponding dicarboxylic acid which comprises isolating a yeast POX4 gene promoter; isolating a target gene involved in dicarboxylic acid production; operably linking the yeast POX4 gene promoter to the open reading frame (ORF) of the target gene involved in dicarboxylic acid production to create a fusion gene; inserting the fusion gene into an expression vector; transforming a yeast host cell with the expression vector; and culturing the transformed yeast host cell in a media containing an organic substrate that is biooxidizable to a mono- or polycarboxylic acid. Preferably, the target gene encodes a member of an ω-hydroxylase complex such as any of the CYP, NCP, or CYTb5 genes.

Preferably, the method of increasing conversion of a fatty acid to its corresponding dicarboxylic acid comprises isolating a promoter from a yeast gene which is induced when the yeast is grown on a fatty acid or alkane substrate; isolating at least one of a CYP, a CYTb5 gene, or a NCP gene; operably linking the inducible gene promoter to the open reading frame (ORF) of at least one of a CYP gene, a CYTb5 gene, or an NCP gene to create a fusion gene; inserting the fusion gene into an expression vector; transforming a yeast host cell with the expression vector; and culturing the transformed host cell in a media containing an organic substrate that is biooxidizable to a mono- or polycarboxylic acid. Preferably, the promoter is the POX4 promoter or else a promoter isolated from a C. tropicalis gene which is induced when the yeast is grown on fatty acids or alkanes. Examples of such yeast genes include but are not limited to: catalase, citrate synthase, 3-ketoacyl-CoA thiolase A, citrate synthase, O-acetylhomoserine sulphydrylase, protease, carnitine O-acetyltransferase, hydratase-dehydrogenase, or epimerase genes. Examples of organic substrates useful in the methods of the present invention, include e.g., a saturated fatty acid, an unsaturated fatty acid, an alkane, an alkene, an alkyne, or a combination thereof.

In still another aspect of the invention, there is provided a method of increasing conversion of a fatty acid to its corresponding dicarboxylic acid which comprises isolating a yeast POX4 gene promoter; isolating at least one of a CYP gene, a CYTb5 gene, or a NCP gene; operably linking the POX4 gene promoter to the open reading frame (ORF) of at least one of a CYP gene, a CYTb5 gene, or an NCP gene to create a fusion gene; inserting the fusion gene into an expression vector; transforming a yeast host cell with the expression vector; and culturing the transformed host cell in a media containing an organic substrate that is biooxidizable to a mono- or polycarboxylic acid. Examples of organic substrate useful in the methods of the present invention, include e.g., a saturated fatty acid, an unsaturated fatty acid, an alkane, an alkene, an alkyne, or a combination thereof.

Examples of host cells which may be used in the above-described methods include e.g., cells from Yarrowia, Candida, Bebaromyces, Saccharomyces, Schizosaccharomyces, or Pichia. Preferably, a host cell is from Candida. Even more preferably, the host cell is from C. tropicalis, C. maltosa, C. apicola, C. paratropicalis, C. albicans, C. cloacae, C. guillernondii, C. intermedia, C. lipolytica, C. parapsilosis or C. zeylenoides. Examples of host cells which may be used in the process include cells from Yarrowia, Candida, Bebaromyces, Saccharomyces, Schizosaccharomyces, or Pichia. Preferably, a host cell is from Candida. Even more preferably, the host cell is from C. tropicalis, C. maltosa, C. apicola, C. paratropicalis, C. albicans, C. cloacae, C. guillermondii, C. intermedia, C. lipolytica, C. parapsilosis or C. zeylenoides. In a more preferred embodiment, the host cell is from C. tropicalis Most preferably, the yeast host cell is from a β-oxidation blocked strain of C. tropicalis.

### DESCRIPTION OF THE DRAWINGS

FIG. 1 shows PCR screening of PR transformants. Lane 1: 100 bp ladder; Lanes 2-11: PR transformants; Lane 12: Blank, and Lane 6: pPR as positive control. Lanes 3 and 6 showed the desired PCR product.

FIG. 2 shows a Southern blot of Pac I digested genomic DNA from PA5 strains screened by using POX4 promoter as the probe. Lanes 4 and 5 show positive hybridization with a band at the expected size.

FIG. 3 shows a Southern blot of Pac I digested genomic DNA from PA2 and PR strains probed with the POX4 promoter. Lane 1: H5343; Lane 2: PA2-2 1; Lane 3: PA2-43; Lane 4: PA2-48; Lane 5: PR-12; and Lane 6: PR-15.

FIG. 4 depicts the nucleotide sequence of a POX4 promoter-CYP52A2A gene fusion construct.

FIG. 5 depicts the nucleotide sequence of a POX4 promoter-CYP52A5A gene fusion construct.

FIG. 6 depicts the nucleotide sequence of a POX4 promoter-NCP1B gene fusion construct.

FIG. 7 graphically depicts reductase activities for yeast strains transformed with POX4 promoter-NCP fusions compared to HDC10-2 (strain with additional copies of native reductase genes) and HDC29-3.

FIG. 8 shows a Western blot using reductase-specific antibody. Heavier band intensities correlating with higher reductase induction is apparent for the POX4-NCP1B transformed yeast strain (PR 12).

FIG. 9 shows average integrated density of different yeast proteins observed as spots on a 2 dimensional gel, under both induced and uninduced conditions.

### DETAILED DESCRIPTION OF THE INVENTION

Increasing dicarboxylic acid production in yeast in accordance with the present invention is based on isolating a promoter from a yeast gene having a desired level of expression and operably linking the promoter to a target gene involved in dicarboxylic acid production. Accordingly, promoter substitution using highly inducible heterologous promoters operably linked to the open reading frame (ORF) of a target gene involved in dicarboxylic acid production in yeast increases the yield of dicarboxylic acids as a result of increased transcription. Furthermore, promoters of gene(s) that are induced at various defined times during the bioconversion in response to certain stimuli (e.g., stress, substrate, cell death) may be utilized for promoter substitution of the target gene(s) thereby leading to increased dicarboxylic acid production at defined times during the bioprocess.

The POX4 gene of C. tropicalis 20336 has been cloned and its DNA sequence determined. Okasaki, K., et al. 1986, PNAS, USA 83:1232-1236. As described above, the POX4 gene encodes a distinct subunit of long chain acyl-CoA oxidase, i.e., the peroxisomal polypeptide (PXP) designated PXP4. In accordance with the present invention, the promoter of the POX4 gene has been determined to be a good candidate for promoter substitution. The POX4 gene is strongly induced in C. tropicalis when grown on fatty acids or alkanes. The promoter has two OLE (oleic acid response element) sequences and no upstream repressive sequences. Since POX4 is not involved in the omega-oxidation pathway, its regulation is different from P450 and reductase promoters.

Any gene involved in fatty acid bioconversion which transcribes at a rate lower than POX4 may be upregulated by the substitution of its native promoter with the POX4 promoter. In a preferred embodiment, the promoter of a CYP, NCP or CYTb5 gene is substituted with the promoter of the Candida tropicalis POX4 or other POX4 gene(s), thereby increasing the transcriptional induction of a CYP, NCP, or CYTb5 gene. As an example, the POX4 promoter may be derived from the POX4 gene of C. tropicalis. The complete promoter of the POX4 gene or a portion thereof containing all of the essential functional sites for the promoter region is operably linked to the open reading frame of a CYP gene, such as e.g., a CYP52A2A, CYP52A5A or NCP1 gene (see U.S. Pat. No. 6,331,420) from C. tropicalis. This in turn results in the increased transcription and production of a CYP or NCP protein and a corresponding increase in the conversion of a fatty acid, e.g., oleic acid, to its corresponding dicarboxylic acid. The term “operably linked” refers to the association of nucleic acid sequences so that the function of one is affected by the other. A promoter is operably linked with an open reading frame when it is capable of affecting the expression of the open reading frame (ORF) (i.e., the ORF is under the transcriptional control of the promoter). Notwithstanding the presence of other sequences between the promoter and ORF, it should be understood that a promoter may still be considered operably linked to the ORF.

In another preferred embodiment the promoter of the CYTb5 (described in U.S. patent application Ser. No. 09/91 1,781, the disclosure of which is incorporated herein as if fully set forth; is replaced by the promoter of the POX4 gene in essentially the same manner described herein, resulting in increased production of the CYTb5 protein and an increase in the conversion of fatty acids to their corresponding dicarboxylic acids. Preferably, the promoter of a P450 gene such as CYP52A2A, CYP52A5A, and/or the reductase gene NCP1 is substituted with a POX4 promoter.

In one embodiment of the present invention, the desired promoter region is isolated using conventional techniques known to those skilled in the art. The POX4 gene or other inducible gene is cut at a convenient location downstream of the promoter terminus using an appropriate restriction enzyme to effect excision. The coding sequence of the POX4 gene or other inducible gene is then removed, to leave essentially a DNA sequence containing the promoter region. For the upstream cutting, a site is selected sufficiently far upstream to include in the retained portion all of the necessary functional sites for the promoter region, and then cut using an appropriate restriction enzyme. Examples of other inducible genes include but are not limited to: catalase, citrate synthase, 3-ketoacyl-CoA thiolase A, citrate synthase, O-acetylhomoserine sulphydrylase, protease, carnitine O-acetyltransferase, hydratase-dehydrogenase, or epimerase genes.

A POX4 promoter or promoter from another inducible gene may also be obtained from a genomic clone via in vitro mutagenesis. There are various commercially available kits particularly suited for this application such as the T7-Gen in vitro Mutagenesis Kit (USB, Cleveland, Ohio) and the QuikChange Site Directed Mutagenesis Kit (Stratagene, San Diego, Calif.). Alternatively, PCR primers can be defined to allow direct amplification of a POX4 promoter.

It should be understood that in all embodiments described herein, a POX4 promoter may be included on a nucleic acid fragment that is larger than the actual promoter region and that the entire fragment, including additional nucleic acid sequence can be utilized for fusion to a target gene.

Next, a promoter/target gene open reading frame nucleotide fusion construct is prepared. The promoter is operably linked to a heterologous target gene, i.e., to the open reading frame of a gene other than that from which the promoter is obtained, to create a nucleotide fusion construct for integration into a host cell. Procedures for fusing promoters to target genes such that they are operably linked and yield the desired DNA construct are well known in the art. Restriction enzymes, ligating enzymes and polymerases are conventional tools commonly utilized by those skilled in the art to create fusion constructs. In a preferred embodiment, polymerase chain reaction (PCR) primers are constructed to amplify the promoter of the POX4 gene using PCR. The correct sequence is verified by conventional techniques known to those skilled in the art. The open reading frame (ORF) and 3′ untranslated region (UTR) of the target gene, e.g. CYP52A2A, CYP52A5A, CYTb5, and/or the reductase gene NCP1, may also be amplified by PCR and verified by sequencing. These two sequences are then fused together by PCR using the two PCR products and the original primers of the initial PCRs that are not homologous at the fusion junction. The product contains the POX4 promoter, the target gene ORF and 3′ UTR and may be confirmed by sequence analysis. If desired, a heterologous 3′ UTR may be used, e.g., a POX4 3′ UTR or other 3′ UTR.

The promoter/target gene ORF fusion constructs are then utilized to create a DNA integration vector for transformation into any suitable host cells. For example, suitable yeast host cells for use in accordance with the present invention include, but are not limited to, Yarrowia, Bebaromyces, Saccharomyces, Schizosaccharomyces, and Pichia and more preferably those of the Candida genus. Preferred species of Candida are tropicalis, maltosa, apicola, paratropicalis, albicans, cloacae, guillermondii, intermedia, lipolytica, parapsilosis and zeylenoides.

Particularly preferred hosts include C. tropicalis strains that have been genetically modified so that one or more of the chromosomal POX4A, POX4B and both POX5 genes have been disrupted as described, e.g., in U.S. Pat. Nos. 5,254,466 and 5,620,878, each incorporated herein by reference. Such disruption blocks the β-oxidation pathway. Examples of β-oxidation blocked strains of C. tropicalis include H41, H41B, H51, H45, H43, H53, H534, H534B, H435 and H5343 (ATCC 20962) as described in aforementioned U.S. Pat. No. 5,254,466.

The DNA constructs described herein may be cloned and expressed in suitable expression vectors. Examples include, but are not limited to vectors such as plasmids, phagemids, phages or cosmids, yeast episomal plasmids, yeast artificial chromosomes, and yeast replicative plasmids. Host cells may also be transformed by introducing into a cell a linear DNA vector(s) containing the desired gene sequence. Such linear DNA may be advantageous when it is desirable to avoid introduction of non-native (foreign) DNA into the cell. For example, DNA consisting of a desired target gene(s) flanked by DNA sequences which are native to the cell can be introduced into the cell by methods such as, but not limited to electroporation, lithium acetate transformation, and spheroplasting. Flanking DNA sequences can include selectable markers and/or other tools for genetic engineering. Yeast cells may be transformed with any of the expression vectors described herein. The term “expression vector” is used broadly herein and is intended to encompass any medium which includes nucleic acid and which can be used to transform a target cell. Expression vectors thus encompass all the examples of vectors listed herein including, e.g., integration vectors.

In a preferred embodiment the DNA construct is used to transform a yeast cell, e.g., a cell of Candida sp., to obtain increased expression therein of a protein, e.g., a CYP or NCP protein, the DNA construct comprising an inducible POX4 promoter DNA for promoter transcription in yeast operably linked to DNA coding for the CYP or NCP protein, to enable expression thereof in the yeast cell. Once created, a yeast host cell containing the POX4 promoter/target gene ORF chimera is generated.

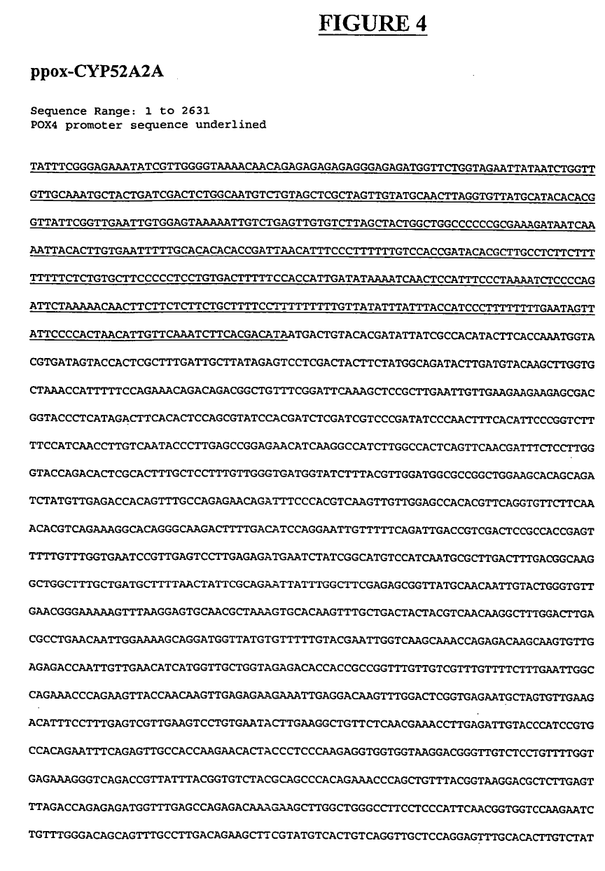

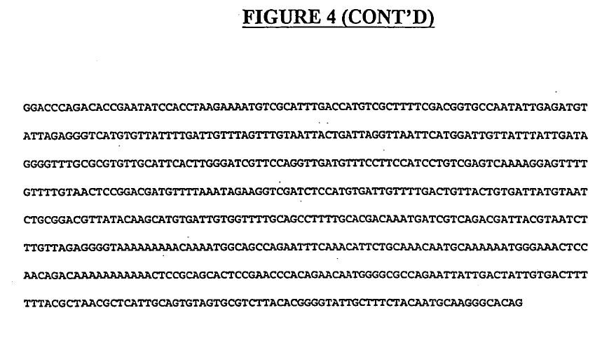

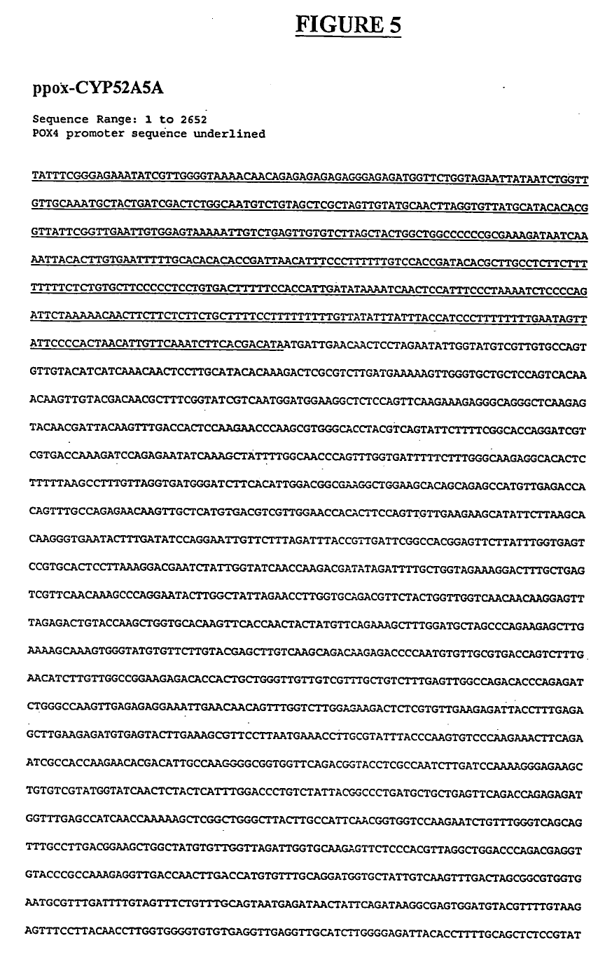

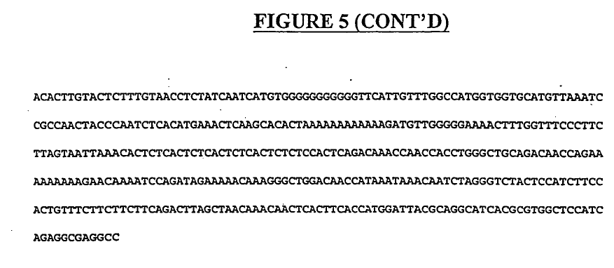

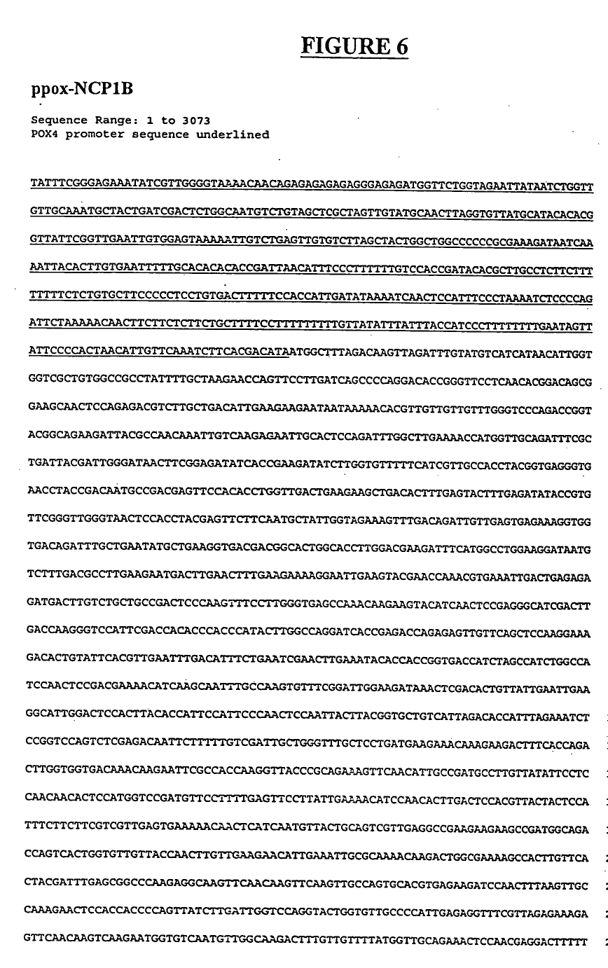

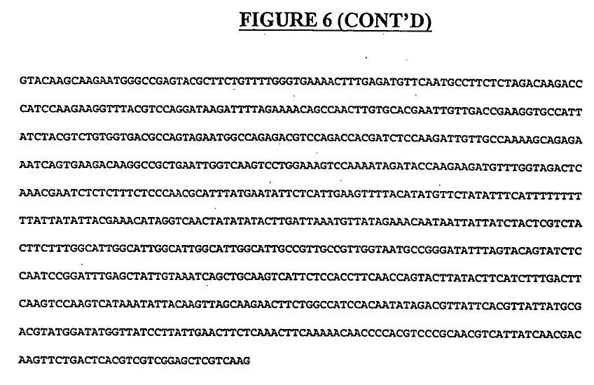

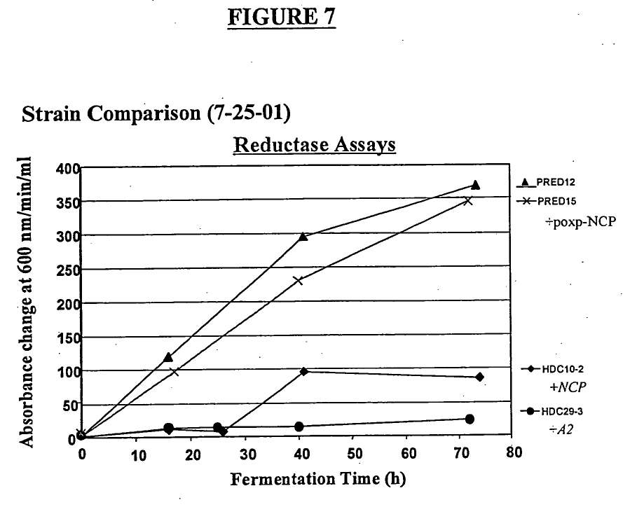

As an example, the complete POX4 promoter or a portion thereof derived from the POX4 gene of C. tropicalis containing all of the essential functional sites for the promoter region is fused to the open reading frame of a CYP52A2A gene from C. tropicalis FIG. 4 depicts the nucleotide sequence of a POX4 promoter-CYP52A2A gene fusion construct. In FIG. 4, the POX4 promoter sequence is underlined. As another example, the complete POX4 promoter or a portion thereof derived from the POX4 gene of C. tropicalis containing all of the essential functional sites for the promoter region is fused to the open reading frame of a CYP52A5A gene. FIG. 5 depicts the nucleotide sequence of a POX4 promoter-CYP52A5A gene fusion construct. In FIG. 5, the POX4 promoter sequence is underlined. As still another example, the complete POX4 promoter or a portion thereof derived from the POX4 gene of C. tropicalis containing all of the essential functional sites for the promoter region is fused to the open reading frame of an NCP gene such as the NCP1B gene. FIG.6 depicts the nucleic acid sequence of a POX4 promoter-NCP1B gene fusion construct. In FIG. 6, the POX4 promoter sequence is underlined.

The strength of the promoter may be measured using techniques well known to those skilled in the art. In a preferred embodiment, promoter strength may be measured using quantitative competitive reverse transcription polymerase chain reaction (QC-RT-PCR) to measure CYP, CYTb5, or NCP gene expression in yeast e.g., Candida cells isolated from fermentors. Enzymatic assays and antibodies specific for CYP, CYTb5, and NCP proteins may be used when appropriate to verify that increased promoter strength is reflected by increased synthesis of the corresponding protein. Diacid productivity is thus improved by selective integration, amplification, and over-expression of CYP, CYTb5 or NCP genes in a yeast production host, e.g., C. tropicalis, C. maltosa, Pichia, etc.

The yeast cells transformed with one of the aforementioned vectors, may be cultured in media containing an organic substrate, to provide improved production of dicarboxylic acid(s). Culturing the yeast, i.e., fermenting the yeast, may be accomplished by procedures well known in the art as described, e.g., in aforesaid U.S. Pat. No. 5,254,466, which disclosure is incorporated by reference herein as if fully set forth.

A suitable organic substrate herein may be any organic compound that is biooxidizable to a mono- or polycarboxylic acid. Such a compound may be any saturated or unsaturated aliphatic compound or any carboxylic or heterocyclic aromatic compound having at least one terminal methyl group, a terminal carboxyl group and/or a terminal functional group which is oxidizable to a carboxyl group by biooxidation. A terminal functional group which is a derivative of a carboxyl group may be present in the substrate molecule and may be converted to a carboxyl group by a reaction other than biooxidation. For example, if the terminal group is an ester that neither the wild-type C. tropicalis nor the genetic modifications described herein will allow hydrolysis of the ester functionality to a carboxyl group, then a lipase can be added during the fermentation step to liberate free fatty acids. Suitable organic substrates include, but are not limited to, saturated fatty acids, unsaturated fatty acids, alkanes, alkenes, alkynes and combinations thereof.

Alkanes are a type of saturated organic substrate which are particularly useful herein. The alkanes can be linear or cyclic, branched or straight chain, substituted or unsubstituted. Particularly preferred alkanes are those having from about 4 to about 25 carbon atoms, examples of which include, but are not limited to, butane, hexane, octane, nonane, dodecane, tridecane, tetradecane, hexadecane, octadecane and the like.

Examples of unsaturated organic substrates which may be used herein include, but are not limited to, internal olefins such as 2-pentene, 2-hexene, 3-hexene, 9-octadecene and the like; unsaturated carboxylic acids such as 2-hexenoic acid and esters thereof, oleic acid and esters thereof including triglyceryl esters having a relatively high oleic acid content, erucic acid and esters thereof including triglyceryl esters having a relatively high erucic acid content, ricinoleic acid and esters thereof including triglyceryl esters having a relatively high ricinoleic acid content, linoleic acid and esters thereof including triglyceryl esters having a relatively high linoleic acid content; unsaturated alcohols such as 3-hexen-1-ol, 9-octadecen-1-ol and the like; unsaturated aldehydes such as 3-hexen-1-al, 9-octadecen-1-al and the like. In addition to the above, an organic substrate which may be used herein include alicyclic compounds having at least one internal carbon-carbon double bond and at least one terminal methyl group, a terminal carboxyl group and/or a terminal functional group which is oxidizable to a carboxyl group by biooxidation. Examples of such compounds include, but are not limited to, 3,6-dimethyl, 1,4-cyclohexadiene, 3-methylcyclohexene, 3-methyl-1, 4-cyclohexadiene and the like.

Examples of the aromatic compounds that may be used herein include but are not limited to, arenes such as o-, m-, p-xylene; o-, m-, p-methyl benzoic acid; dimethyl pyridine, sterols and the like. The organic substrate can also contain other functional groups that are biooxidizable to carboxyl groups such as an aldehyde or alcohol group. The organic substrate can also contain other functional groups that are not biooxidizable to carboxyl groups and do not interfere with the biooxidation such as halogens, ethers, and the like.

Examples of saturated fatty acids which may be applied to yeast cells incorporating the aforementioned fusion constructs according to the present invention include caproic, enanthic, caprylic, pelargonic, capric, undecylic, lauric, myristic, pentadecanoic, palmitic, margaric, stearic, arachidic, behenic acids and combinations thereof. Examples of unsaturated fatty acids which may be applied to genetically modified yeast cells include palmitoleic, oleic, erucic, linoleic, linolenic acids and combinations thereof. Alkanes and fractions of alkanes may be applied which include chain links from C12 to C24 in any combination. An example of a preferred fatty acid mixture is HOSFFA (high oleic sunflower oil, i.e., fatty acid mixture containing approximately 80% oleic acid commercially available from Cognis Corp. as Edenor®).

The invention is further illustrated by the following specific examples which are not intended in any way to limit the scope of the invention.

### EXAMPLE 1

### POX4 Promoter Amplification

The POX4 promoter-gene fusion fragment was constructed by cloning the promoter and gene fragments separately and then fused together using high fidelity PCR. All PCR cloning reactions were carried out using Expand High Fidelity PCR reagent from Roche.

The POX4 genomic clone pKD3 was sequenced using sequencing primer POX4pr (CAACCGAATAACCGTGTG) (SEQ ID NO: 1). The upstream sequence of −932 to −1 (+1 being the translational start codon of the POX4 gene) from the POX4 promoter region was obtained.

The POX4 promoter was amplified by high fidelity PCR using an universal forward primer (PoxPacI) with the Pac I restriction site added paired with reverse primers with matching sequences to that of the genes that will be fused with the promoter. The primers are listed below:

TABLE 1
Primers used to amplify gene specific POX4 promoter

| | |
|---|---|
| Primer Name | Primer Sequences |

| | | |
|---|---|---|
| PoxPac I | TTAATTAA TATTTCGGGAGAAATATCGTTGGGG | (SEQ ID NO:2) |
| A2POXR | GATAATATCGTGTACAGTCATTATGTCGTGAAGATTTGA | (SEQ ID NO:3) |
| A5POXR | TTCTAGGAGTTGTTCAATCATTATGTCGTGAAGATTTGA | (SEQ ID NO:4) |
| REDPOXR | ATCTAACTTGTCTAAAGCCATTATGTCGTGAAGATTTGA | (SEQ ID NO:5) |

The PCR amplified fragment was labeled as POXP-_{A2}, POXP-_{A5}, and POXP-_{RED} respectively and was used in the promoter-gene fusion high fidelity PCR reactions.

### EXAMPLE 2

### CYP52A2, CYP52A5 and NCP1 gene amplification

The ORF (open reading frame) and the downstream region of CYP52A2, CYP52A5 and NCP1 were amplified by high fidelity PCR. The following primers were used in the PCR reactions:

TABLE 2
Primers used to amplify ORFs of CYP52A2, CYP52A5 and NCP1

| | |
|---|---|
| Primer Name | Primer Sequences |

| | | |
|---|---|---|
| PoxA2F | TCAAATCTTCACGACATAATGACTGTACACGATATTATC | (SEQ ID NO:6) |
| A2Pac | TTAATTAA CTGTGCCCTTGCATTGTAG | (SEQ ID NO:7) |
| PoxA5F | TCAAATCTTCACGACATAATGATTGAACAACTCCTAGAA | (SEQ ID NO:8) |
| A5Pac | TTAATTAA GGCCTCGCCTCTGATGGAG | (SEQ ID NO:9) |
| PoxRedF | TCAAATCTTCACGACATAATGGCTTTAGACAAGTTAGAT | (SEQ ID NO:10) |
| RedPac | TTAATTAA CTTGACGAGCTCCGACGAC | (SEQ ID NO:11) |

CYP52A2 was amplified using PoxA2F and A2Pac primers and the PCR product was named pox-A2. CYP52A5 was amplified using PoxA5 and A5Pac primers and the PCR product was named pox-A5. NCP1 was amplified using PoxRedF and RedPac primers and the PCR product was named pox-Red. These PCR products were cloned into pCR2.1 using Invitrogen's TOPO cloning kit and transformed into DH5α. Positive transformants were screened by PCR and plasmids were prepared using the Qiagen miniprep kit. The plasmid harboring the pox-A2, pox-A5, and pox-Red fragment was named TA/A2, TA/A5, and TA/Red, respectively.

TABLE 3

Primers used to verify DNA sequences of PCR
amplified CYP52A2, CYP52A5 and NCP1 genes

| | | |
|---|---|---|
| Gene | Primer Name | Sequence |

| | | |
|---|---|---|
| CYP52A2A | M13 forward | GTAAAACGACGGCCAG |
| | | (SEQ ID NO:12) |
| | M13 reverse | CAGGAAACAGCTATGAC |
| | | (SEQ ID NO:13) |
| | A2-ORF1 | AGAAAGGCACAGGGCAAGAC |
| | | (SEQ ID NO:14) |
| | A2-ORF2 | TGCCACCAAGAACACTACCC |
| | | (SEQ ID NO:15) |
| CYP52A5A | M13 forward | GTAAAACGACGGCCAG |
| | | (SEQ ID NO:16) |
| | M13 reverse | CAGGAAACAGCTATGAC |
| | | (SEQ ID NO:17) |
| | A5-ORF1 | TCTTATTTGGTGAGTCCGTGC |
| | | (SEQ ID NO:18) |
| | A5-ORF2 | CCCAAGAAACTTCAGAATCGC |
| | | (SEQ ID NO:19) |
| | A5-ORF3 | TACAACCTTGGTGGGGTGTGTG |
| | | (SEQ ID NO:20) |
| NCP1B | M13 reverse | CAGGAAACAGCTATGAC |
| | | (SEQ ID NO:21) |
| | RED-ORF1 | TATGCTGAAGGTGACGACGG |
| | | (SEQ ID NO:22) |
| | RED-ORF2 | TGCTGGGTTTGCTCCTGATG |
| | | (SEQ ID NO:23) |
| | RED-ORF3 | CCCCATTGAGAGGTTTCGTTAG |
| | | (SEQ ID NO:24) |
| | RED-ORF4 | GAATCTCTCTTTCTCCCAACGC |
| | | (SEQ ID NO:25) |

### EXAMPLE 3

### POX4 Promoter-Gene Fusion

The promoter and gene fragment were fused together using high fidelity PCR. The POX4 promoter—A2 fusion was amplified using PoxPacI and A2Pac as primers and POXP-_{A2} and TA/A2 as template. The pox promoter—A5 fusion was amplified using PoxPacI and A5Pac as primers and POXP-_{A5} and TA/A5 as template. The pox promoter—NCP fusion was amplified using PoxPacI and RedPac as primers and POXP-_{RED} and TA/Red as template.

TABLE 4
Primers and templates used to amplify promoter-gene fusions

| Fusion Name | Promoter | Gene | Primer | Template |
|---|---|---|---|---|
| ppoxCYP52A2 | POX4 | CYP52A2 | PoxPacI | POXP-_{A2} |
| | | | A2Pac | TA/A2 |
| ppoxCYP52A5 | POX4 | CYP52A5 | PoxPacI | POXP-_{A5} |
| | | | A5Pac | TA/A5 |
| ppoxNCP | POX4 | NCP1 | PoxPacI | POXP-_{RED} |
| | | | RedPac | TA/Red |

These PCR products were gel-purified and cloned into pCR2.1 using Invitrogen's TOPO cloning kit. Positive transformants were screened by PCR using primer POXPF paired with A2R, A5R, or RedR primer.

TABLE 5

Sequences of primers used to screen for
positive transformants containing fusion
constructs

| | |
|---|---|
| Primer Name | Sequence |

| | | |
|---|---|---|
| POXPF | TTTTTTTCTCTGTGCTTCCCCC | (SEQ ID NO:26) |
| A2R | ATCGTGGATACGCTGGAGTGTG | (SEQ ID NO:27) |
| A5R | AACTTGTTCTCTGGCAAACTGTGG | (SEQ ID NO:28) |
| RedR | AACTCGTCGGCATTGTCGGTAG | (SEQ ID NO:29) |

The TOPO cloned ppoxA2, ppoxA5 and ppoxRed plasmid were named pPA2, pPA5, and pPRed, respectively. These plasmids were sequenced (Sequentech) to ensure no mutations were introduced during the PCR cloning process.

PPA2, pPA5, and pPRed were digested by Pac I to release the ppox-gene fusion fragment. These Pac I fragments were ligated with Pac I opened pURAin, a pNEB 193 based plasmid with inverted URA3 fragments. The resulting pURAin ligated POX4 promoter and gene fusion plasmid were designated pUPA2, pUPA5, and pUPRed, respectively. The following table provides an index for the naming history.

TABLE 6
A list of names used in the fusion strain construction

| | CYP52A2 | CYP52A5 | NCP1 |
|---|---|---|---|
| PCR pox promoter | POX-_{A2} | POX-_{A5} | POX-_{Red} |
| PCR gene fragment | pox-A2 | pox-A5 | pox-Red |
| TA cloned gene | PTA/A2 | Pta/A5 | pTA/Red |
| Fusion fragment | _{PPOX}CYP52A2 | _{PPOX}CYP52A5 | _{PPOX}NCP1 |
| Fusion in pCR2.1 | PPA2 | pPA5 | pPRed |
| Fusion in pURAin | PUPA2 | pUPA5 | pUPRed |
| Fusion strains | PA2 | PA5 | PR |

### EXAMPLE 4

### Construction of Promoter Substituted Strain

The transformation fragments were released from pUPA2, pUPA5, and pUPRed using Asc I and Pme I double digest. The fragments were used to transform C. tropicalis strains H5343 ura- or HDC100 ura- to obtained promoter substituted strains. Candida transformation was carried out by the lithium acetate protocol. Briefly, 10-20 ml of YEPD was inoculated with the base strain (H5343 ura- or HDC100 ura-, a beta-oxidation blocked strain) and grown overnight at 30° C. with agitation. The O.D.₆₀₀ was measured and 100 ml of YEPD was inoculated such that the O.D. would be ˜0.6 in about 2 generations. Cells were harvested by centrifugation at 6000 g for 5 minutes. Cells were washed once in 20 ml of sterile distilled and deionized water and resuspended in 10 ml LiSORB (100 mM LiOAC, 10 mM Tris, pH 8, 1 mM EDTA, 1 M Sorbitol). After incubation at 30° C. for 15-30 minutes, cells were spun down as described above and resuspend in 125 μl LiSORB. Samples were then held on ice. 10 μg of transforming DNA was mixed with 50 μl of LiSORB. 100 μl of the cells were mixed to the DNA mix. 1.8 ml of 40% PEG in 100 mM LiOAC/TE (100 mM LiOAC, 10 mM Tris pH 8, 1 mM EDTA) were added and the cell mixture allowed to incubate at 30° C. for 30 minutes. Cells were heat shocked at 42° C. for 14 minutes. Cells were plated on Ura dropout plates and incubated at 30° C. for 3-5 days for colonies to grow.

### EXAMPLE 5

### Screening Transformants

Transformants were initially screened by PCR and positive transformants were confirmed by Southern blot analysis. The primers used in the PCR screening are listed below.

TABLE 7
Primers used to screen fusion transformants

| | |
|---|---|
| Primer Name | Sequence |

| | | |
|---|---|---|
| Poxp-For | TTTTTTTCTCTGTGCTTCCCCC | (SEQ ID NO:30) |
| A2-Rev | ATCGTGGATACGCTGGAGTGTG | (SEQ ID NO:31) |
| A5-Rev | AACTTGTTCTCTGGCAAACTGTGG | (SEQ ID NO:32) |
| Red-Rev | AACTCGTCGGCATTGTCGGTAG | (SEQ ID NO:33) |

Positive transformants screened by PCR were further analyzed by Southern blot. The ECL direct nucleic acid labelling and detection systems kit from Amersham was used and the manufacture's suggested protocol was followed.

TABLE 8
List of selected POX4 promoter-gene fusion strains

| Strain Name | ppoxGene fusion |
|---|---|
| PR12 | ppoxNCP1 |
| PR15 | ppoxNCP1 |
| PA5-17 | ppoxCYP52A5 |
| PA5-20 | ppoxCYP52A5 |
| PA2-21 | ppoxCYP52A2 |
| PA2-43 | ppoxCYP52A2 |

### EXAMPLE 6

### Filamentous Growth Test

All the new promoter substitution strains were tested for growth rate and filamentous growth. Strains were grown overnight in half strength YEPD in a 37° C. shaker. The overnight cultures were examined under microscope to determine whether the strains were grown filamentous. Only non-filamentous strains were used in further evaluations in the mini-fermentor system (Model Sixfors, Infors Ag, Bottmingen, Switzerland).

### EXAMPLE 7

### Reductase Activities

Reductase activity showed the pox-gene fusion strains (PR12 and PR15) had significant higher reductase activity over the base strain (H5343) and the strain with additional copies of native reductase genes (HDC10).

The reductase assay was conducted in a reaction mixture (1.0 ml) containing 100 mM HEPES, pH 7.6, 0.12 mM NADPH, 32 mM nicotinamide, 50 μM cytochrome C, 0.25 mM sodium cyanide, and cell extract (dilution was carried out as needed to give a linear rate over one minute of reaction time). The reaction was initiated by addition of NADPH. Enzyme activity was measured spectrophotometrically by following the increase in absorbance at 550 nm.

### EXAMPLE 8

### Western Blot

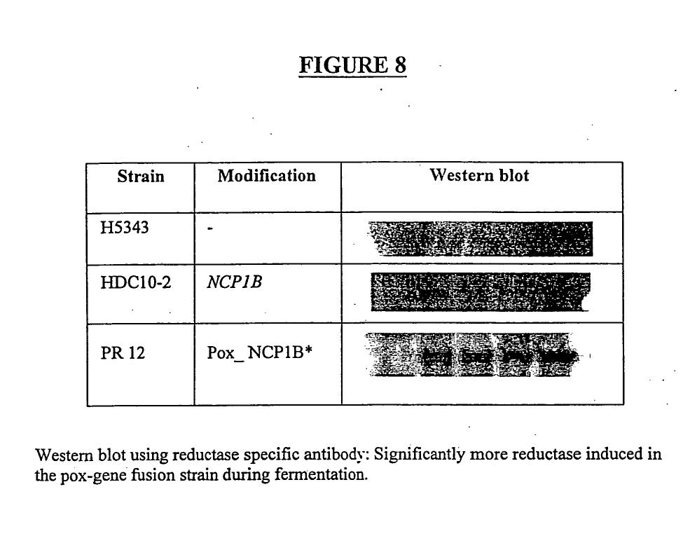

The levels of induced NCP protein were measured in different yeast strains. NCP protein was detected and measured by Western Blot using a reductase specific antibody. Candida tropicalis strain H5343 showed bands correlating to reductase. HDC10-2, a C. tropicalis strain modified with an additional reductase gene, showed higher levels of reductase enzyme compared to H5343. PR12, which is a Candida tropicalis strain genetically modified with the POX4promoter-NCP1B gene showed significant increase in reductase induction (FIG. 8).

The reductase antibody was obtained using recombinant antibody technology. An antigenic peptide (SEDKAAELVKSWKVQNRYQEDVW) (SEQ ID NO:34) was selected from the C-terminal of the deduced amino acid sequence of the NCP1 gene. The synthetic peptide was conjugated to a carrier protein and injected into sheep to generate antibody. The IgG fractions were purified and evaluated against the antigenic peptide and the recombinant reductase protein expressed in E. coli. The antibody was found specific to the reductase protein with a sensitivity of 2 ng at 10,000× dilution. Western blot was carried out according to standard protocol known to those skilled in the art.

### EXAMPLE 9

### Results

Although there were two approaches originally designed to make the promoter-gene fusion, the PCR cloning approach proved advantageous due to its speed and high fidelity. All fusion constructs were made without errors and subsequently cloned into the transformation vector pURAin. The URA3 flanked fusion construct was released by AscI/PmeI digest and used in transforming C. tropicalis base strains. PA2 and PR strains were made by transforming H5343 ura-strain to obtain PA5. PA5 strains were made by transforming HDC100 ura-strain. A high frequency of URA3 reversion was encountered when PA5 strains were attempted by transforming H5343 ura-.

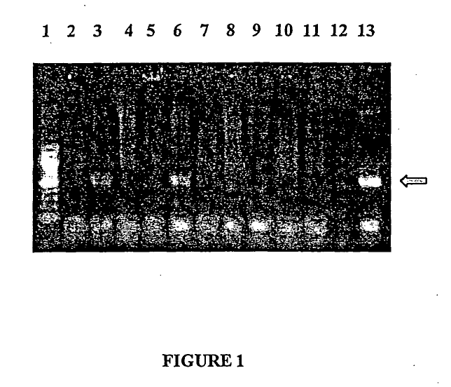

The promoter-gene fusion construct was made using high fidelity PCR and the sequence was verified by DNA sequencing. The PacI fragment of the fusion construct was cloned into the PacI site of pURAin. The transformation vector was released from the plasmid after AscI/PmeI digest. Transformants were screened initially by PCR (FIG. 1).

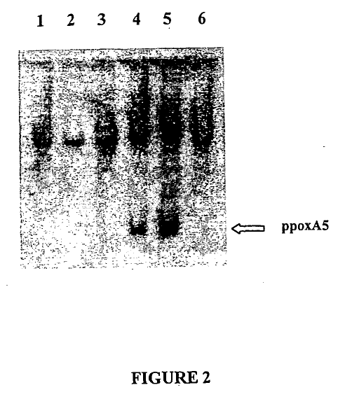

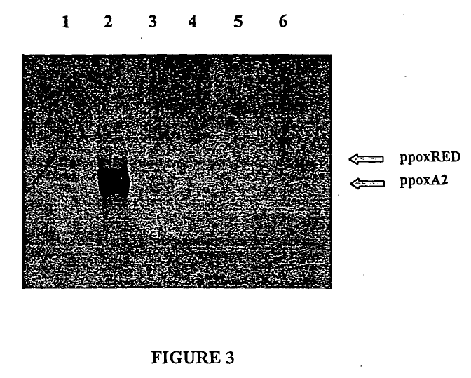

Positive transformants from the PCR screening were further analyzed and confirmed by Southern blot analysis (FIGS. 2 and 3). Two PA2 strains (PA2-21 and PA2-43), ten PA5 strains (PA5-7, PA5-9, PA5-14, PA5-17, PA5-20, PA5-23, PA5-27, PA5-28, and PA5-30) were obtained. Comparing the intensity of the hybridization band with that of the native POX4 promoter band, all the strains have 1-2 copies of the fusion construct except PA21, which has probably more than 10 copies of the ppoxA2 construct. More analysis is needed to accurately determine the copy number of the fusion construct in the strains. None of the strains were filamentous when grown in half strength YEPD at 37° C.

TABLE 9
Summary of Productivity Results for Best
Strains - HOSFFA as Substrate

| | Genetic | Productivity* | Improvement |
|---|---|---|---|
| Strain | Modifications | (g/kg/h) | over H5343 (%) |
| H5343 | — | 0.940 | 0.00 |
| HDC10 | NCP1B | 1.095 | 16.51 |
| PRED12 | ppoxNCP1B | 1.149 | 22.29 |
| HDC15 | CYP52A5A | 0.907 | −3.48 |
| PA5 | ppoxCYP52A5A | 1.055 | 12.26 |

The results showed that the pox-gene fusion strains had significantly higher product conversion rate. The fusion strains also showed improvements over additional native gene incorporation.

Two ppox-A2 strains (PA2-21 and PA2-43) and two ppox-NCP strains (PRED12 and PRED15) were obtained by transforming H5343. There were some difficulties in screening ppox-A5 transformants when H5343 was used as the host strain. However, ten ppox-A5 strains (PA5-7, PA5-9, PA5-14, PA5-17, PA5-20, PA5-22, PA5-23, PA5-27, PA5-28, and PA5-30) were obtained when HDC100 was used as the host strain. None of the pox-gene fusion strains were filamentous when grown overnight in half strength YEPD in a 37° C. shaker. Promoter-gene fusion strains PA2-21, PA2-43, PA5-17, PA5-20, PRED12 and PRED15 were tested in the mini fermentor and samples were taken for QC-RT-PCR, Western blot, and GC analysis. The PA2 strains did not show significant difference from H5343 in CYP52A2 gene induction, enzyme production, nor dicarboxylic acid conversion. The PR strains showed significant improvement over H5343 in terms of enzyme quantity and activity, and dicarboxylic acid conversion. The PA5 strains showed some improvement in the diacid conversion.

### EXAMPLE 10

Since it has been determined that the POX4 promoter is induced even when the POX4 gene is disrupted, experiments were performed to identify other Candida genes which have inducible promoters which may be used in the methods of the present invention. Thus, proteins were isolated from C. tropicalis strain HDC23-3 grown either in the presence of, or without, fatty acids, in order to examine the induction characteristics, if any, of such proteins. In particular, the cell growth media was HOSFFA (high oleic sunflower oil). Cells were removed at time 0 (uninduced) and after eight (8) hours (induced) of growth in HOSFFA. Cells were lysed, and proteins extracted following standard protocols. Protein samples were electrophoresed on a 2D gel and spots selected for further characterization. In-gel digests of the proteins were performed and the protein identification was obtained using MALDI MS.

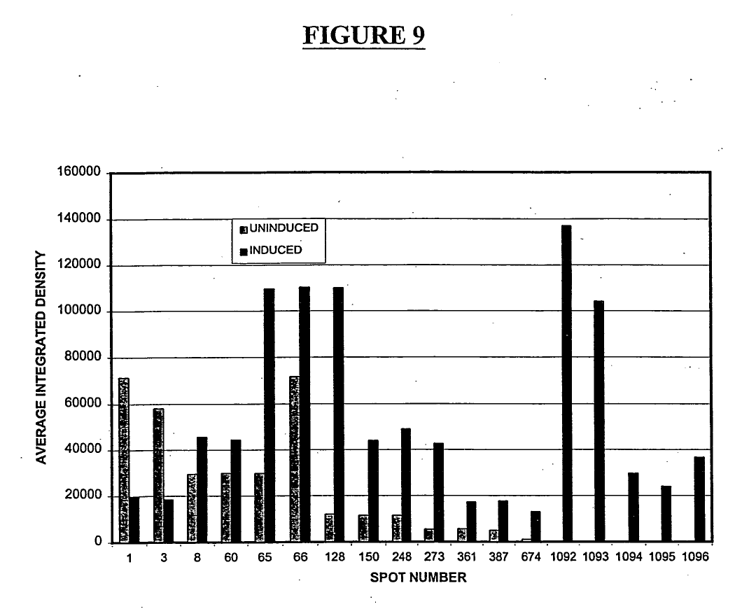

Table 10 lists those enzymes correlating to specific spots on the 2D gel. The spot numbers and average integrated density for uninduced and induced proteins correlated to the average integrated density shown in FIG. 9.

TABLE 10

| | | | | Fold |
|---|---|---|---|---|
| Enzyme Name | SPOT # | UNINDUCED | INDUCED | Induction |
| Catalase | 65 | 29860 | 109580 | 3.7 |
| Citrate synthase | 66 | 71780 | 110280 | 1.5 |
| Catalase | 128 | 12080 | 109970 | 9.1 |
| 3-ketoacyl-CoA thiolase A | 150 | 11490 | 43940 | 3.8 |
| Citrate synthase | 248 | 11530 | 48910 | 4.2 |
| O-acetylhomoserine sulphydrylase | 273 | 5460 | 42580 | 7.8 |
| | 361 | 5690 | 17280 | 3.0 |
| Yeast protease | 387 | 5120 | 17730 | 3.5 |
| Carnitine O-acetyltransferase | 674 | 1200 | 13220 | 11 |
| Hydratase-dehydrogenaes epimerase | 1092 | 0 | 136970 | |
| | 1093 | 0 | 104080 | |
| | 1094 | 0 | 29760 | |
| | 1095 | 0 | 23990 | |
| Citrate synthase | 1096 | 0 | 36697 | |

*0 means the corresponding spot had a value non-differentiable to the background.

Summary of 2D-MS/MS Analysis Results

There were 13 spots sent for identification and 10 of 13 were identified via MALDI MS analysis with significant matches to known Candida tropicalis proteins in the databases. Results are shown in Table 11.

TABLE 11

| Spot Number | Protein Name |
|---|---|
| MSN 65 MSN 128 | catalase |
| MSN 66 MSN 248 MSN 1096 | citrate synthase |
| MS 150 | 3-ketoacyl-CoA thiolase A (pot1/pox3?) |
| MSN 273 | o-acetylhomoserine sulphydrylase |
| MS 674 | carnitine o-acetyltransferase |
| MSN 1092 MSN 1093 | hydratase-dehydrogenase epimerase |

Most of the proteins identified are located in the mitochondria and peroxisome. Naturally, these proteins are strongly induced by fatty acids and involved in fatty acid metabolism (beta-oxidation or oxidative metabolism). The induction of beta-oxidation enzymes does not necessarily mean beta-oxidation is active, since a key enzyme (products of POX genes) in the pathway is missing.

None of the p450s were among the major spots. A mitochondrial 2D preparation may be needed in order to clear the background so that P450 will be among the major spots.

There were signs that some of the proteins were degrading, either within the cell or during sample preparation. Spots 1092 and 1093 both showed streaking lines on the 2D gel and both were identified to the same protein. There were three spots (66, 248, 1096) identified as citrate synthase, with the most abundant spot (66) having the highest MW and pH.

Several candidate clones carrying partial cDNAs for yeast peroxisomal enzymes, such as catalase, carnitine acetyltransferase, isocitrate lyase, malate synthase and acyl-CoA oxidase, have been reported as efficiently isolated at a single plating from a phage lambda gt11 recombinant cDNA library. The library was prepared with poly(A)-rich RNA from an n-alkane-grown yeast, Candida tropicalis, and probed with a mixture of antibodies against the respective purified enzymes. See Ueda M, Okada H, Hishida T, Teranishi Y, and Tanaka A, “Isolation of several cDNAs encoding yeast peroxisomal enzymes” FEBS Lett Aug. 10, 1987; 220(1):31-5.

The corresponding genomic clones to these cDNAs may be obtained by screening a genomic library using the cDNAs, portions thereof, or oligonucleotide sequences corresponding thereto, as probes. Once the genomic clones are obtained, they may be characterized by mapping and/or partial or complete sequencing in order to locate the 5′ upstream regulatory regions. Such regions comprise inducible promoters which may be isolated using well known procedures for use in the methods of the present invention.

What is claimed is:

1. A method of converting a fatty acid to its corresponding dicarboxylic acid which comprises:
(a) isolating a yeast POX4 gene promoter;
(b) isolating a target gene involved in dicarboxylic acid production;
(c) operably linking the yeast POX4 gene promoter to the open reading frame (ORF) of the target gene involved in dicarboxylic acid production to create a fusion gene;
(d) inserting the fusion gene into an expression vector;
(e) transforming a yeast host cell with the expression vector; and
(f) culturing the transformed yeast host cell in a media containing an organic substrate that is biooxidizable to a mono- or polycarboxylic acid.

2. A method for transforming a yeast host cell, said method comprising:
(a) isolating a POX4 promoter;
(b) isolating a target gene;
(c) operably linking a POX4 promoter to the open reading frame of the target gene to create a fusion gene;
(d) inserting the fusion gene into an expression vector; and
(e) transforming the host cell with the expression vector.

3. The method of claim 2 wherein the native POX4 gene of the host cell is disrupted or deleted.

4. The method of claim 1 wherein the target gene codes for a member of an ω-hydroxylase complex.

5. The method of claim 4 wherein the target gene encoding a member of an ω-hydroxylase complex is a CYP, NCP, or CYTb5 gene.

6. The method of claim 5 wherein the CYP, NCP, or CYTb5 gene is selected from the group consisting of CYP52A2A, CYP52A5A, NCP1B, or CYTb5 genes.

7. The method of claim 2 wherein the target gene encodes a member of an ω-hydroxylase complex.

8. The method of claim 7 wherein the target gene coding for a member of an ω-hydroxylase complex is a CYP, NCP, or CYTb5 gene.

9. The method of claim 8 wherein the CYP, NCP, or CYTb5 genes are selected from the group consisting of CYP52A2A, CYP52A5A, NCP1B, or CYTb5 genes.

10. A host cell comprising a nucleic acid molecule for a POX4 gene promoter operably linked to the open reading frame of a gene encoding a heterologous protein.

11. The host cell of claim 10 wherein the gene encoding a heterologous protein encodes a member of an ω-hydroxylase complex such as any of the CYP, NCP, or CYTb5 genes.

12. The host cell of claim 11 wherein the CYP, NCP, or CYTb5 genes are selected from the group consisting of CYP52A2A, CYP52A5A, NCP1B, or CYTb5 genes.

13. The host cell of claim 10 selected from the group consisting of Yarrowia, Candida, Bebaromyces, Saccharomyces, Schizosaccharomyces, and Pichia.

14. The Candida host cell of claim 13 selected from the group consisting of C. tropicalis, C. maltosa, C. apicola, C. paratropicalis, C. albicans, C. cloacae, C. guillermondii, C. intermedia, C. lipolytica, C. parapsilosis, and C. zeylenoides.

15. The Candida host cell of claim 14 wherein the host cell is C. tropicalis.

16. The host cell of claim 15 wherein the host cell is from a β-oxidation blocked strain of C. tropicalis.

17. A method of converting a fatty acid to its corresponding dicarboxylic acid, said method comprising:
(a) isolating a promoter from a yeast gene which is induced when the yeast is grown on fatty acids or alkanes;
(b) isolating a target gene involved in dicarboxylic acid production;
(c) operably linking the inducible gene promoter to the open reading frame (ORF) of the target gene involved in dicarboxylic acid production to create a fusion gene;
(d) inserting the fusion gene into an expression vector;
(e) transforming a yeast host cell with the expression vector; and
(f) culturing the transformed yeast host cell in a media containing an organic substrate that is biooxidizable to a mono- or polycarboxylic acid.

18. The method of claim 17 wherein the promoter is the POX4 promoter.

19. The method of claim 17 wherein the promoter is isolated from a C. tropicalis gene which is induced when the yeast is grown on fatty acids or alkanes.

20. The method of claim 17 wherein the isolated promoter is from a C. tropicalis catalase, citrate synthase, 3-ketoacyl-CoA thiolase A, citrate synthase, O-acetylhomoserine sulphydrylase, protease, carnitine O-acetyltransferase, hydratase-dehydrogenase, or epimerase gene.

21. The method of claim 17 wherein the target gene encodes a member of an ω-hydroxylase complex such as any of the CYP, NCP, or CYTb5 genes.

22. The method of claim 21 wherein the CYP, NCP, or CYTb5 genes are selected from the group consisting of CYP52A2A, CYP52A5A, NCP1B, or CYTb5 genes.

23. A method for increasing conversion of a fatty acid to its corresponding dicarboxylic acid, said method comprising:
(a) isolating a promoter from a yeast gene which is induced when the yeast is grown on a fatty acid or alkane substrate;
(b) isolating at least one of a CYP, a CYTb5 gene, or a NCP gene;
(c) operably linking the inducible gene promoter to the open reading frame (ORF) of at least one of a CYP gene, a CYTb5 gene, or an NCP gene to create a fusion gene;
(d) inserting the fusion gene into an expression vector;
(e) transforming a yeast host cell with the expression vector; and
(f) culturing the transformed host cell in a media containing an organic substrate that is biooxidizable to a mono- or polycarboxylic acid.

24. The method of claim 23 wherein the promoter is the POX4 promoter.

25. The method of claim 23 wherein the promoter is isolated from a C. tropicalis gene which is induced when the yeast is grown on fatty acids or alkanes.

26. The method of claim 23 wherein the promoter is from a gene selected from the group consisting of catalase, citrate synthase, 3-ketoacyl-CoA thiolase A, citrate synthase, O-acetylhomoserine sulphydrylase, protease, carnitine O-acetyltransferase, hydratase-dehydrogenase, or epimerase genes.

27. The method of claim 23 wherein the organic substrate is a saturated fatty acid, an unsaturated fatty acid, an alkane, an alkene, an alkyne, or a combination thereof.


SEQUENCE LISTING (34 sequences)

SEQ ID NO: 1 - DNA, 18 bp, Artificial Sequence
  primer
caaccgaataaccgtgtg

SEQ ID NO: 2 - DNA, 33 bp, Artificial Sequence
  primer
ttaattaatatttcgggagaaatatcgttgggg

SEQ ID NO: 3 - DNA, 39 bp, Artificial Sequence
  primer
gataatatcgtgtacagtcattatgtcgtgaagatttga

SEQ ID NO: 4 - DNA, 39 bp, Artificial Sequence
  primer
ttctaggagttgttcaatcattatgtcgtgaagatttga

SEQ ID NO: 5 - DNA, 39 bp, Artificial Sequence
  primer
atctaacttgtctaaagccattatgtcgtgaagatttga

SEQ ID NO: 6 - DNA, 39 bp, Artificial Sequence
  primer
tcaaatcttcacgacataatgactgtacacgatattatc

SEQ ID NO: 7 - DNA, 27 bp, Artificial Sequence
  primer
ttaattaactgtgcccttgcattgtag

SEQ ID NO: 8 - DNA, 39 bp, Artificial Sequence
  primer
tcaaatcttcacgacataatgattgaacaactcctagaa

SEQ ID NO: 9 - DNA, 27 bp, Artificial Sequence
  primer
ttaattaaggcctcgcctctgatggag

SEQ ID NO: 10 - DNA, 39 bp, Artificial Sequence
  primer
tcaaatcttcacgacataatggctttagacaagttagat

SEQ ID NO: 11 - DNA, 27 bp, Artificial Sequence
  primer
ttaattaacttgacgagctccgacgac

SEQ ID NO: 12 - DNA, 16 bp, Artificial Sequence
  primer
gtaaaacgacggccag

SEQ ID NO: 13 - DNA, 17 bp, Artificial Sequence
  primer
caggaaacagctatgac

SEQ ID NO: 14 - DNA, 20 bp, Artificial Sequence
  primer
agaaaggcacagggcaagac

SEQ ID NO: 15 - DNA, 20 bp, Artificial Sequence
  primer
tgccaccaagaacactaccc

SEQ ID NO: 16 - DNA, 16 bp, Artificial Sequence
  primer
gtaaaacgacggccag

SEQ ID NO: 17 - DNA, 17 bp, Artificial Sequence
  primer
caggaaacagctatgac

SEQ ID NO: 18 - DNA, 21 bp, Artificial Sequence
  primer
tcttatttggtgagtccgtgc

SEQ ID NO: 19 - DNA, 21 bp, Artificial Sequence
  primer
cccaagaaacttcagaatcgc

SEQ ID NO: 20 - DNA, 22 bp, Artificial Sequence
  primer
tacaaccttggtggggtgtgtg

SEQ ID NO: 21 - DNA, 17 bp, Artificial Sequence
  primer
caggaaacagctatgac

SEQ ID NO: 22 - DNA, 20 bp, Artificial Sequence
  primer
tatgctgaaggtgacgacgg

SEQ ID NO: 23 - DNA, 20 bp, Artificial Sequence
  primer
tgctgggtttgctcctgatg

SEQ ID NO: 24 - DNA, 22 bp, Artificial Sequence
  primer
ccccattgagaggtttcgttag

SEQ ID NO: 25 - DNA, 22 bp, Artificial Sequence
  primer
gaatctctctttctcccaacgc

SEQ ID NO: 26 - DNA, 22 bp, Artificial Sequence
  primer
tttttttctctgtgcttccccc

SEQ ID NO: 27 - DNA, 22 bp, Artificial Sequence
  primer
atcgtggatacgctggagtgtg

SEQ ID NO: 28 - DNA, 24 bp, Artificial Sequence
  primer
aacttgttctctggcaaactgtgg

SEQ ID NO: 29 - DNA, 22 bp, Artificial Sequence
  primer
aactcgtcggcattgtcggtag

SEQ ID NO: 30 - DNA, 22 bp, Artificial Sequence
  primer
tttttttctctgtgcttccccc

SEQ ID NO: 31 - DNA, 22 bp, Artificial Sequence
  primer
atcgtggatacgctggagtgtg

SEQ ID NO: 32 - DNA, 24 bp, Artificial Sequence
  primer
aacttgttctctggcaaactgtgg

SEQ ID NO: 33 - DNA, 22 bp, Artificial Sequence
  primer
aactcgtcggcattgtcggtag

SEQ ID NO: 34 - protein, 23 aa, Artificial Sequence
  peptide derived from C-terminal end of deduced amino acid sequence of NCP1 gene
SEDKAAELVKSWKVQNRYQEDVW